(19) United States
(12) Patent Application Publication
(10) Pub. No.: US 2026/0256994 A1
(43) Pub. Date: Sep. 3, 2026

(54) MEDICAL DEVICE, IN PARTICULAR A STENT

(71) Applicant: ACANDIS GMBH, Pforzheim (DE)

(72) Inventors: David Klopp, Remchingen (DE); Katharina Birkner, Pforzheim (DE); Lilija Ignatjew, Neuhausen (DE)

(21) Appl. No.: 19/140,737

(22) PCT Filed: Dec. 20, 2023
(86) PCT No.: PCT/EP2023/086938

§ 371 Date: Jun. 18, 2025

(30) Foreign Application Priority Data
Dec. 22, 2022 (DE) 10 2022 134 575.0

(51) Int. Cl. A61L 31/10; A61L 31/04; A61L 31/16

(52) U.S. Cl. CPC A61L 31/10; A61L 31/046; A61L 31/16; A61L 2300/414; A61L 2400/12; A61L 2400/18; A61L 2420/02; A61L 2420/06; A61L 2420/08

(57) ABSTRACT

A medical device, in particular a stent, having a self-expandable mesh structure, which is at least partially tubular and is able to widen automatically from a compressed cross-sectional diameter to an expanded cross-sectional diameter, wherein the mesh structure has at least one mesh structure element which is encased, in particular in its entirety, by a nanostructure coating formed of fibrin nanofibers, wherein the nanostructure coating has a first layer, which forms an in particular felt-like or fleece-like matrix of crosslinked fibrin fibers, wherein some fibrin fibers protrude freely above the first layer, and a second layer of an in particular nap-like single-fiber structure, and wherein the nanostructure coating additionally contains a growth factor or a peptide having the functional structure of a growth factor, which growth factor or peptide is integrated in the first layer and/or the second layer.

### CROSS REFERENCE TO RELATED PATENT APPLICATIONS

This is a U.S. national phase patent application of PCT/EP2023/086938 filed Dec. 20, 2023, which claims the benefit of and priority to German Patent Application No. 10 2022 134 575.0, filed on Dec. 22, 2022, the entire contents of each of which are incorporated herein by reference for all purposes.

### TECHNICAL FIELD

The invention relates to a medical device, in particular a stent.

### BACKGROUND

In particular, the document DE 10 2018 110 591 A1 cited above describes a stent which has a biological coating, which promotes the endothelialisation, i.e. the deposition, of endothelial cells onto the stent. The base of the stent forms a self-expandable mesh structure which is at least partially tubular in construction and which is capable of dilating automatically from a compressed cross-sectional diameter to an expanded cross-sectional diameter. The mesh structure has at least one mesh structure element which is encased by a nanostructure coating. This nanostructure coating is formed from fibrin nanofibers.

In order to form the biological coating in the known stent, firstly, fibrinogen is provided and then converted into fibrin by adding thrombin. In this way, the fibrin forms filaments which extend outwards from the surface of the mesh structure. This forms a loose fibrin network into which heparin can be deposited.

### SUMMARY

Although the previously known medical device exhibits good results having regard to endothelialisation, a further improvement would be desirable. Thus, the objective of the present invention is to provide a medical device which achieves a further improvement in endothelialisation.

In accordance with the invention, this objective is achieved by the medical device shown and described herein.

Therefore, the invention is based on the concept of providing a medical device, in particular a stent, with a self-expandable mesh structure which is at least partially tubular in construction and which can be dilated automatically from a compressed cross-sectional diameter to an expanded cross-sectional diameter. The mesh structure has at least one mesh structure element which is encased, in particular completely, by a nanostructure coating which is formed from fibrin nanofibers. In accordance with the invention, the nanostructure coating has a first layer which forms a matrix, in particular a mat-like or fleece-like matrix, produced from fibrin fibers which are interconnected with each other, wherein some fibrin fibers protrude freely over the first layer and form a second layer produced from a single-fiber structure, in particular a pile-like structure. Furthermore, the nanostructure coating additionally contains a growth factor or a peptide with the functional structure of a growth factor, which is integrated into the first layer and/or the second layer. “Integrated” as used here means the fact that the growth factor or a peptide with the functional structure of a growth factor is present in the first layer and/or the second layer in a bonded or unbonded form.

Thus, the invention is distinguished from the previously known prior art in that the nanostructure coating is substantially two-layered in construction and in addition has a growth factor or a peptide with the functional structure of a growth factor. A first layer, which preferably lies directly on the surface of the mesh structure element, has a matrix produced from interconnected fibrin fibers. This matrix is particularly dense. In this respect, the matrix can also be described as being mat-like or fleece-like. In particular, the fibrin fibers of the first layer are interconnected with each other or matted together. This forms a particularly dense fibrin structure.

However, a portion of the fibrin fibers protrudes over the first layer and forms a single-fiber structure. The protruding fibrin fibers are preferably not interconnected in the region of the second layer and, rather, protrude over the first layer as single fibers. These single fibers or single fiber portions essentially form a pile-like structure. Thus, a particularly loose fibrin fiber structure is present in the second layer.

Essentially, the nanostructure coating can be likened to a velour carpet, in which the textile fibers in a first layer are interconnected with each other or matted together and in an overlying layer, individual textile fibers protrude freely and thus form the pile of the velour.

The growth factor or the peptide with the functional structure of a growth factor integrated into the nanostructure coating brings about a further improvement in the cell viability of the medical device, which results in better and more rapid endothelialisation.

In the context of the present invention, the “peptide with the functional structure of a growth factor” designates a peptide which, like a growth factor, is capable of binding to a specific receptor in the cell membrane of a cell the biological function of which is designed and provided for binding to growth factors. Compared with the growth factor, this is therefore a truncated protein fragment or a peptide which has the same amino acid sequence as the relevant sub-region of the growth factor and can bind to relevant receptors, or a peptide which has been synthetically produced by combinatorial methods for binding to relevant receptors.

It has been shown that overall, this particular structure of the nanostructure coating provides an increased surface area for the adhesion of endothelial cells. Endothelialisation is therefore significantly improved. As is known from the prior art, the first layer, which is more strongly interconnected, may have substantially sponge-like properties, so that an increased quantity of heparin can be taken up in the first layer. The heparin improves the anti-thrombogenicity of the nanostructure coating. This does not exclude the heparin from also binding to the second layer. Rather, the heparin can bind to the nanostructure coating over the entire layer thickness.

In principle, the growth factor may be any known growth factor, wherein growth factors selected from the group comprising vascular endothelial growth factors (VEGF), fibroblast growth factors (FGF), epidermal growth factors, growth factors derived from blood platelets, neurotrophins and insulin-like growth factors, can be cited as being particularly suitable. In one embodiment, the peptide with the functional structure of a growth factor is derived from such a growth factor, i.e. it has a structure which has at least 95% and preferably at least 97% identity with a sub-structure of the growth factor, and wherein discrepancies in the identity are formed by conservative substitutions. In this context, “identity” stands for sequence homology and the expression “conservative substitution” stands for the replacement of one amino acid in the sequence by another amino acid the properties of which as regards the electrical charge, the functional groups it contains, the hydrophobicity, and the size, are similar.

In the context of the invention described here, particularly preferred growth factors are vascular endothelial growth factors and fibroblast growth factors, and peptides with the functional structure of such a growth factor as the peptides.

In order to provide improved endothelialisation, advantageously, the growth factor or the peptide with the functional structure of a growth factor is covalently bonded to components of the nanostructure coating. Appropriately, this bonding is by bonding to the fibrin nanofibers of the nanostructure coating. The covalent bonding here may be obtained by any chemical reaction which is compatible with the conditions for the production of the medical device. The bonding may occur directly with the fibrinogen before it is used to form the fibrin nanofilaments, or the growth factor or the peptide with the functional structure of the growth factor may be integrated into them after the production of the nanostructure coating.

In this case, but also in the case of covalent bonding to fibrinogen, the growth factor or the peptide may be appropriately bonded to components of the nanostructure coating via an amino group, by click chemistry or via a thiol group. More particularly preferably, the bonding is the reaction product of a primary amine with a dialdehyde or epoxide, the reaction product of a C—C triple bond with an azide group (generally known as “Click Chemistry”), or the reaction product of a thiol group with an alkene or epoxy group. These reactions have the advantage that as a rule, they are selective and the desired bonding can also take place under aqueous conditions. For the cited reactions, it is not important which of the functional groups is bonded to the growth factor or the peptide with the functional structure of the growth factor, i.e. this or the peptide may, for example, contain a C—C triple bond or an azide group and in the respective cases, a component of the nanostructure coating will contain the respectively complementary group (i.e. an azide group or a C—C triple bond).

In a preferred embodiment of the medical device in accordance with the invention, the nanostructure coating has a quantity of protein of at least 2 μg/cm², in particular more than 2 μg/cm², in particular more than 3 μg/cm². It has been shown experimentally that such a quantity of protein results in a sufficiently dense and simultaneously thin nanostructure coating. A thin nanostructure coating is advantageous in order to keep the total thickness of the nanostructure element in a range such that the entire medical device can be correctly compressed to a cross-sectional diameter which is as small as possible. This is the condition required for the medical device to be capable of being guided to the treatment site via small catheters. Thus, small blood vessels, in particular in the cerebral region, can also be treated.

In addition, with a thin nanostructure coating, the total wall thickness of the device remains small and therefore the flow of blood through a blood vessel is not substantially compromised. Narrowing of the vessel (stenosis) caused by the device is therefore avoided.

Advantageously, in particular, the first layer has a first quantity of protein and the second layer has a second quantity of protein, wherein the first quantity of protein is larger than the second quantity of protein. Specifically, it has been shown that a ratio between the first quantity of protein and the second quantity of protein of at least 2, in particular at least 3, in particular at least 4, is advantageous. This ensures that on the one hand, the first layer is sufficiently dense to form a sponge-like structure in order to integrate heparin or other substances, and on the other hand, the second layer has a sufficiently loose structure in order to ensure improved adhesion of endothelial cells. In fact, heparin can also bond covalently to the second layer and prevent blood clotting there because of its anti-thrombogenic properties. However, it is anticipated that, because of the sponge-like structure therein, a larger quantity of heparin will be deposited in the first layer, which is then gradually given up to the second layer. In this respect, the first layer can also form a reservoir for substances.

In an advantageous embodiment, the first layer in the medical device in accordance with the invention may have a height between 5 nm and 100 nm, in particular between 5 nm and 50 nm, in particular between 5 nm and 30 nm, in particular between 10 nm and 40 nm, in particular between 20 nm and 30 nm. The second layer may have a height between 5 nm and 200 nm, in particular between 5 nm and 100 nm, in particular between 5 nm and 50 nm, in particular between 5 nm and 30 nm, in particular between 10 nm and 40 nm, in particular between 20 nm and 30 nm. In this regard, it is particularly preferable for the total height of the nanostructure coating to be at most 300 nm, in particular at most 200 nm, in particular at most 150 nm, in particular at most 120 nm, in particular at most 100 nm, in particular at most 90 nm, in particular at most 80 nm, in particular at most 60 nm.

A contribution to the stability of the fibrin nanocoating is advantageously made when the fibrin fibers are formed from crosslinked fibrin molecules. The crosslinking here may be obtained by the addition of a particular factor, factor XIIIa, in the production process. The fibrin nanostructure is significantly stabilised by the crosslinking and the aforementioned advantages as regards endothelialisation can be achieved in an improved manner. In respect of the crosslinking, in particular, the fibrin molecules in this regard respectively have two carboxyl termini (D domains) and one amino terminus (E domain), wherein the amino terminus of one fibrin molecule is bonded to at least one carboxyl terminus of another fibrin molecule, in particular by means of a covalent bond.

### DESCRIPTION OF DRAWINGS

The invention will now be described in more detail with the aid of an exemplary embodiment and with reference to the accompanying diagrammatic drawings, in which:

FIG. 1 shows a diagrammatic cross-sectional view through a mesh structure element with a nanostructure coating of a medical device in accordance with the invention, in a preferred exemplary embodiment;

FIG. 2 shows a top view of a nanostructure coating of a mesh structure element of a medical device in accordance with the invention, in a scanning electron microscope image; and

FIG. 3 shows a diagrammatic representation of the crosslinking of fibrin molecules for the formation of a stabilised fibrin fiber;

FIG. 4 shows the comparison of glass surface cell viability for a glass surface modified with fibrin and heparin (Fb+H), a glass surface modified with fibrin, heparin and factor XIII and a glass surface modified with fibrin, heparin, factor XIII, FGF und VEGF.

### DESCRIPTION OF AN EMBODIMENT

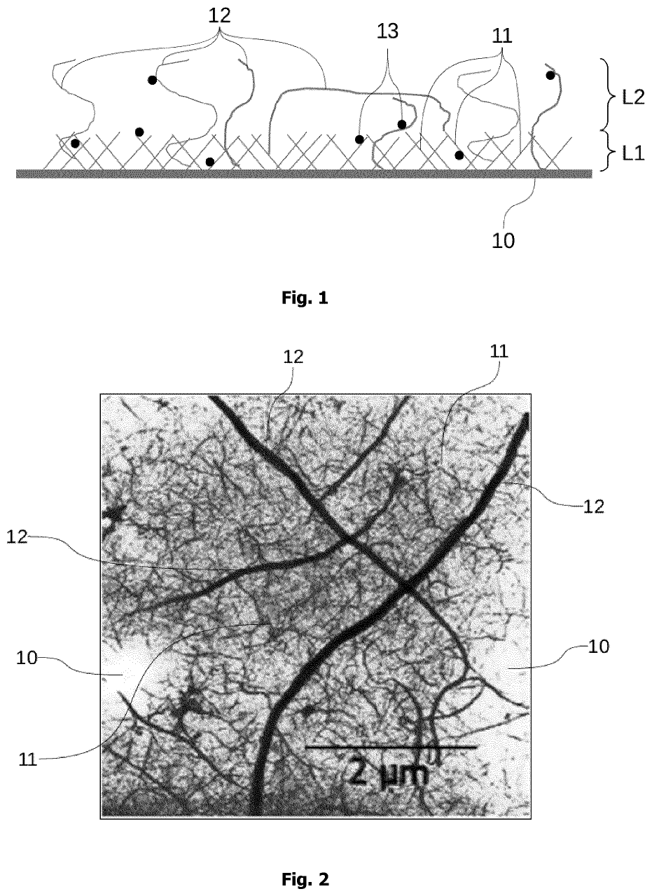

FIG. 1 diagrammatically shows a mesh structure element 10 of a medical device, in particular a stent. In general, in the medical device, a plurality of mesh structure elements 10 are provided which form a mesh structure. Here, the mesh structure elements 10 may be formed by wires which are intertwined with each other in order to form a mesh structure. Alternatively, the mesh structure elements 10 may also form webs of a one-piece mesh structure. This is the case, for example, with stents which are cut in one piece from a tube. Stents of this type are often known as laser-cut stents.

The diagrammatic representation of FIG. 1 substantially shows a cross-section through the mesh structure element 10, wherein, for the sake of clarity, the nanostructure coating is shown on only the top side. The nanostructure coating extends, however, over the entire outer surface of the mesh structure element 10, i.e. the mesh structure element 10 may be completely encased by the nanostructure coating. In particular, the nanostructure coating extends only over the outer circumferential surface of the individual mesh structure elements 10. Cells or meshes of a mesh structure, i.e. openings, which are bordered by the individual mesh structure elements 10, are preferably not covered over by the nanostructure coating. However, advantageously, all of the mesh structure elements 10 of the mesh structure are completely encased by the nanostructure coating.

The nanostructure coating has a first layer L1 which lies directly on the surface of the mesh structure element 10. The first layer L1 is formed by fibrin fibers 11 which form a densely interconnected matrix. As a consequence, the fibrin fibers 11 are interengaged and are very compacted, so that essentially, a fleece-like first layer L1 is present.

The diagrammatic representation of FIG. 1 shows that individual fibrin fibers 12 protrude over the first layer L1 as single fibers. These protruding fibrin fibers 12 form a second layer L2 of the nanostructure coating above the first layer L1. The second layer L2 is formed by a single-fiber structure, i.e. here, the fibrin fibers 12 are present as free fibers, in particular with largely free ends. The second layer L2 therefore substantially forms a pile produced from protruding fibrin fibers 12.

The representation in FIG. 1 can be likened to a velour carpet, in which the mesh structure element 10 forms the base layer. Textile fibers are disposed on the base layer, wherein in a first layer, the textile fibers are interconnected to form a fleece directly on the base layer. Individual fibers of the fleece protrude over the first layer and form a pile produced from single fibers. A similar structure is produced here during the nanostructure coating.

In FIG. 1, growth factor proteins 13 represented by. are covalently bonded to fibrin filaments which are located in the first and second layer.

The structure of the nanostructure coating can be seen in the scanning electron microscope image of FIG. 2. The view shows fibrin fibers 11, 12, which are disposed on a mesh structure element 10. The thickness of the fibrin fibers 11, 12 shown in the view varies. This variation in thickness substantially results from the perspective. This means that the fibrin fibers 12 which appear to be thicker are disposed closer to the microscope lens than the fibrin fibers 11 which appear to be thinner. In other words, the fibrin fibers 12 which appear to be thicker in the scanning electron microscope image protrude further over the outer surface of the mesh structure element 10 than fibrin fibers 11 which appear to be thinner.

FIG. 2 clearly shows that the fibrin fibers 11 disposed closer to the mesh structure element 10 are strongly interconnected and in this respect form the fleece-like first layer L1 of the nanostructure coating. Individual, thicker-appearing fibrin fibers 12 protrude further out, and therefore are at a greater distance from the mesh structure element 10. These individual fibrin fibers 12 are present as single fibers and form the pile-like second layer L2 of the nanostructure coating. FIG. 2 makes it clear that the second layer L2 has a significantly lower fibrin fiber density than the first layer L1.

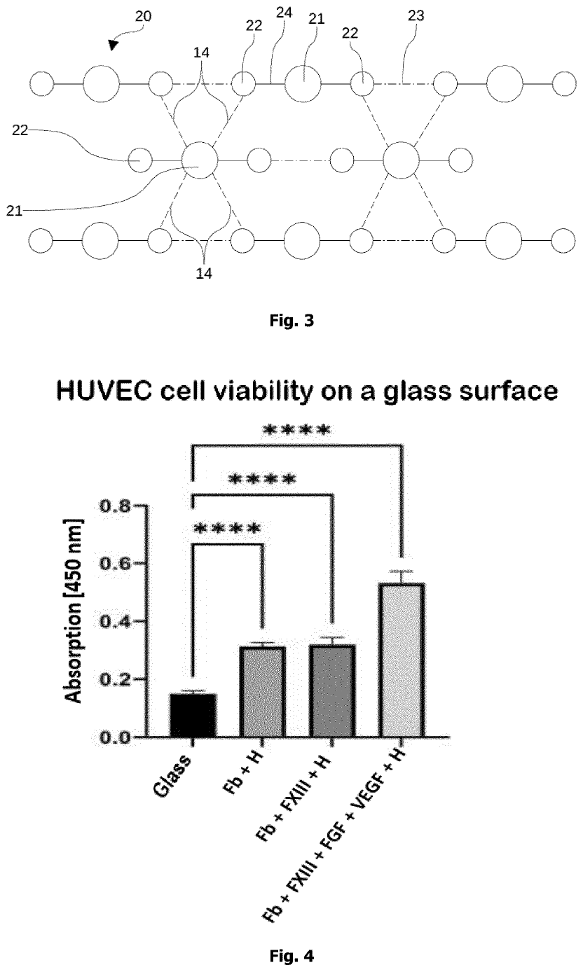

The individual fibrin fibers 11, 12 are formed from fibrin molecules 20, which form a compound by the addition of thrombin, so that the fibrin fibers 11, 12 are formed. In a very simplified representation, as can be seen in FIG. 3, each fibrin molecule 20 has a central amino terminus which is also known as the E domain 21. Individual monomers of what is known as a coiled coil structure 24 extend from the E domain 21. Carboxyl termini are present on the outside of the molecule; they are known as D domains 22.

Fibrin peptides are dissociated by the addition of thrombin, whereupon the fibrin monomers which are released in this manner are interlinked by polymer bonds 23. Crosslinking bonds 14 are formed by the further addition of a fibrin-stabilising factor (factor XIIIa). The crosslinking bonds 14 are formed here between the E domain 21 and respectively at least one D domain 22 of another fibrin molecule 20. In particular, adjacent D domains 22 of two fibrin molecules 20 are bonded to the E domain 21 of a third fibrin molecule 20 by the covalent bonding 14. In this manner, the covalent bonds 14 form a bridge which stabilises the weaker polymerisation bond 23. Overall, the entire fibrin fiber structure is stabilised in this manner.

The nanostructure coating described in connection with the present invention is particularly effective having regard to bonding endothelial cells. In experiments, the nanostructure coating was applied to a glass substrate and immersed with the glass substrate in an endothelial cell solution. It was thus shown that the construction of the nanostructure coating is such that at least 60000, in particular between 60000 and 90000 endothelial cells per square centimetre become deposited on the nanostructure coating. This is a significant increase in the number of endothelial cells per square centimetre compared with previous biological coatings.

Not only is the number of endothelial cells per square centimetre increased with the nanostructure coating of the invention, but also, it has been shown that the cell viability after three days is significantly increased compared with previous biological coatings. Thus, after three days, more endothelial cells have survived on the nanostructure coating of the medical device in accordance with the invention than on other previously known biological coatings. Specifically, the cell viability after three days was determined experimentally by means of a CCK assay on a stent with the nanostructure coating described here, wherein an absorbance of significantly more than 0.2 was observed at a wavelength of 450 nm.

In FIG. 4, the cell viability of Human Umbilical Vein Endothelial Cells (HUVEC) on a glass surface without a coating and on glass surfaces which had been modified with various coatings was investigated. In the case of a coating with fibrin and heparin, a significantly improved viability was observed; crosslinking the fibrin with fibrin-stabilising factor (factor XIII) was not pertinent to the viability, but inhibited a simple detachability of the fibrin film from the surface. By the addition in accordance with the invention of FGF and VEGF, on the other hand, the cell viability could be significantly increased once again. This result shows that a significantly improved endothelialisation can be expected for a relevant surface modification.

### LIST OF REFERENCE NUMERALS

10 mesh structure element
11 fibrin fibers of fiber matrix
12 fibrin fibers of single-fiber structure
13 growth factor
14 covalent bond
20 fibrin molecule
21 E domain
22 D domain
23 polymeric bond
24 coiled coil structure
LI first layer
L2 second layer

1. A medical device with a self-expandable mesh structure which is at least partially tubular in construction and which can be dilated automatically from a compressed cross-sectional diameter to an expanded cross-sectional diameter, wherein the mesh structure has at least one mesh structure element which is encased by a nanostructure coating which is formed from fibrin nanofibers, wherein the nanostructure coating has a first layer which forms a matrix produced from fibrin fibers which are interconnected with each other, wherein some of the fibrin fibers protrude freely over the first layer and form a second layer produced from a single-fiber structure, and wherein the nanostructure coating additionally contains a growth factor or a peptide with a functional structure of a growth factor which is integrated into the first layer and/or the second layer.

2. The medical device according to claim 1, wherein the growth factor or the peptide with the functional structure of a growth factor is covalently bonded to components of the nanostructure coating.

3. The medical device according to claim 2, wherein the bonding of the growth factor or of the peptide with the functional structure of a growth factor to components of the nanostructure coating is obtained via an amino group, by click chemistry or via a thiol group.

4. The medical device according to claim 3, wherein the growth factor is selected from a group comprising vascular endothelial growth factors, fibroblast growth factors, epidermal growth factors, growth factors derived from blood platelets, neurotrophins and insulin-like growth factors, and the peptide with the functional structure of a growth factor is derived from such a growth factor.

5. The medical device according to claim 1, wherein the nanostructure coating has a quantity of protein of at least 2 μg/cm².

6. The medical device according to claim 1, wherein the first layer has a first quantity of protein and the second layer has a second quantity of protein, wherein a ratio between the first quantity of protein and the second quantity of protein is at least 2.

7. The medical device according to claim 1, wherein the second layer has a greater height than the first layer.

8. The medical device according to claim 1, wherein the first layer has a height between 5 nm and 100 nm, and the second layer has a height between 5 nm and 200 nm.

9. The medical device according to claim 1, wherein the fibrin fibers are formed from crosslinked fibrin molecules.

10. The medical device according to claim 9, wherein the fibrin molecules respectively have two carboxyl termini (D domains) and one amino terminus (E domain), wherein the amino terminus of a first fibrin molecule is bonded to at least one carboxyl terminus of a second fibrin molecule.

11. The medical device according to claim 1, wherein the medical device is a stent.

12. The medical device according to claim 1, wherein the matrix is a mat-like or a fleece-like matrix.

13. The medical device according to claim 1, wherein the single-fiber structure is a pile-like structure.

14. The medical device according to claim 2, wherein the growth factor is covalently bonded to fibrin nanofibers of the nanostructure coating.

15. The medical device according to claim 3, wherein the bonding is a reaction product of a reaction of a primary amine with a dialdehyde or an epoxide, a reaction product of a C—C triple bond with an azide group, or a reaction product of a thiol group with an alkene or an epoxy group.

16. The medical device accruing to claim 10, wherein the amino terminus of the first fibrin molecule is bonded to the at least one carboxyl terminus of the second fibrin molecule by means of a covalent bond.
